(19) United States
(12) Patent Application Publication
(10) Pub. No.: US 2022/0119337 A1
(43) Pub. Date: Apr. 21, 2022

(54) PROCESS FOR PREPARATION OF HALOGENATED BENZYLAMINE AND INTERMEDIATES THEROF

(71) Applicant: SRF LIMITED, Gurgaon (IN)

(72) Inventors: Amardeep SINGH, Gurgaon (IN); Bhupender SINGH, Gurgaon (IN); Ram SINGH, Gurgaon (IN); Rajender KUMAR, Gurgaon (IN); Ajay Kumar CHAUDHARY, Gurgaon (IN); Kapil KUMAR, Gurgaon (IN); Anurag JAIN, Gurgaon (IN)

(21) Appl. No.: 17/422,385

(22) PCT Filed: Jan. 23, 2020
(86) PCT No.: PCT/IN2020/050072

§ 371 Date: Jul. 12, 2021

(30) Foreign Application Priority Data
Jan. 25, 2019 (IN) 201911003239
Jan. 31, 2019 (IN) 201911003810

(51) Int. Cl. C07C 209/48

(52) U.S. Cl. CPC C07C 209/48

(57) ABSTRACT

The present invention provides an improved process for the preparation of halogenated benzylamine having the formula I from halogenated benzonitriles, Formula I wherein, X₁ is selected from group consisting of hydrogen, chloro or fluoro, provided at least one X₁ is chloro or fluoro.

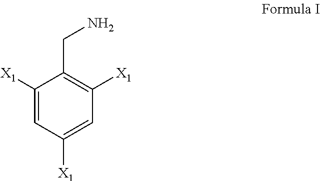

### FIELD OF THE INVENTION

The present invention provides an improved process for preparation of halogenated benzylamine of formula I from halogenated benzonitriles,

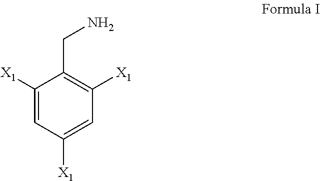

wherein, X₁ is selected from hydrogen, chloro and fluoro, provided at least one X₁ is chloro or fluoro.

### BACKGROUND OF THE INVENTION

Halogenated benzylamine derivatives are widely used as raw material for pharmaceutical intermediates, agrochemicals, drugs, dyes etc. It is an intermediate for N-(2,4,6-trifluorobenzoyl)-N′-[3,5-dichloro-4-(3-chloro-5-trifluoromethyl-2-pyridyloxy) phenyl]urea, useful for controlling various kinds of insect pests. Various methods are known in the art for preparation of halogenated benzyl amine.

Chinese Patent No. 106349083 provides a process for preparation of 2,4,6-trifluorobenzylamine using 2,4,6-trifluorobenzonitrile in presence of Raney nickel and aqueous ammonia. It has been observed by present inventors that addition of aqueous ammonia in the 2,4,6-trifluorobenzonitrile in presence of Raney nickel affects the yield and selectivity of product.

Japanese Patent No. 2993877 provides a process for preparation of 2,4,6-trifluorobenzonitrile using 3,5-dichloro-2,4,6-trifluorobenzonitrile in presence of zinc as metal catalyst and concentrated sulphuric acid in benzonitrile at 130° C. Sulphuric acid is highly reactive and results in high reaction temperature leading to degradation and decomposition of product thereby releasing hydrogen, which is a safety concern at commercial scale. The benzonitrile used as a solvent in this process, produces benzoic acid and amide by-products thereby requiring an extra effort to isolate the product of high purity.

Thus, there is an urgent need to develop an economic, high yielding, safe and robust process for preparation of halogenated benzylamines. The inventors of present invention have carried out multiple experiments to improve the process for preparation of halogenated benzylamine

### OBJECT OF THE INVENTION

The present invention provides an improved and selective process for preparation of halogenated benzylamine from halogenated benzonitriles and also provides a robust process of selective dehalogenation of halogenated benzonitrile.

### SUMMARY OF THE INVENTION

In first aspect, the present invention provides an improved process for preparation of halogenated benzylamine of formula I, comprising the steps of:

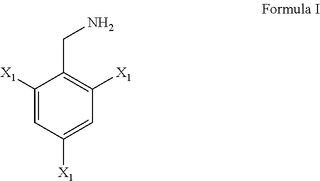

wherein, X₁ is selected from hydrogen, chloro and fluoro, provided at least one X₁ is chloro or fluoro.

a) selective dehalogenation of halogenated benzonitrile of formula III,

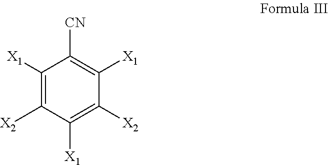

wherein, X₁ is selected from hydrogen, chloro and fluoro, provided at least one X₁ is chloro or fluoro; X₂ is selected from chloro or bromo;

using a transition metal catalyst in presence of an alkanoic acid to obtain halogenated benzonitrile of formula II; and

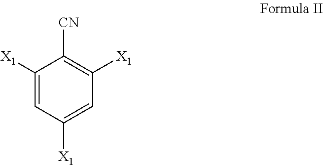

wherein, X₁ is selected from hydrogen, chloro or fluoro, provided at least one X₁ is chloro or fluoro.

b) hydrogenating halogenated benzonitrile of formula II using hydrogenating catalyst and ammonia in presence of a solvent to obtain halogenated benzylamine of formula I.

The second aspect of the present invention provides a process for preparation of halogenated benzylamine of formula I comprising a step of hydrogenating halogenated benzonitrile of formula II being carried out by continuous addition of halogenated benzonitrile of formula II to a mixture of hydrogenating catalyst, ammonia and a solvent.

### DETAILED DESCRIPTION OF THE INVENTION

As used herein, dehalogenation refers to replacement of halogen group with hydrogen group and preferably debromination and dechlorination.

As used herein, continuous addition refers to a slow addition or dropwise addition for the present invention.

In an embodiment, dehalogenation of halogenated benzonitrile of formula III is carried out using a transition metal catalyst in presence of alkanoic acid to obtain halogenated benzonitrile of formula II.

As used herein, the term “alkanoic acid” is selected from formic acid, acetic acid, trifluoroacetic acid, or the like. The molar ratio of alkanoic acid with respect to the halogenated benzonitrile of formula III is in the range of 2-5.

In an embodiment, alkanoic acid is added continuously in the dehalogenation reaction in 2-5 hours. The present inventors observed an improvement in the selectivity by carrying out continuous addition of alkanoic acid in the reaction mixture.

As used herein, the “transition metal catalyst” is selected from copper, zinc, zinc/copper alloy or the like. The transition metal catalyst for dehalogenation process is used in solid or powder form. The transition metal catalyst contains a metal content greater than 99%. The mesh size of metal powder used is less than 100 micron, more preferably between 2-50 microns and most preferably between 2-20 microns.

In an embodiment, 3,5-dichloro-2,4,6-trifluorobenzonitrile is dechlorinated using zinc powder of mesh size between 5-10 micron in presence of acetic acid.

The dehalogenation step of present invention involves two dehalogenation step to form a halogenated benzonitrile of formula II from halogenated benzonitrile of formula III. In a case, where only one dehalogenation step takes place, the intermediate formed refers to compound of formula IV, is isolated and recycled to dehalogenation step.

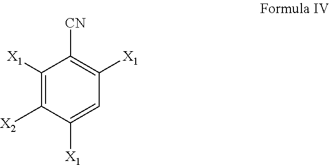

wherein, X₁ is selected from hydrogen, chloro or fluoro, provided at least one X₁ is chloro or fluoro; X₂ is chloro or bromo.

The reaction temperature for dehalogenation ranges from 70° C. to 90° C. The lower temperature range prevents the degradation of product and improves yield significantly. The impurities in the dehalogenation step of halogenated benzonitrile of formula III are formed by dehalogenation of X₁ selected from chloro or fluoro, from compound of formula III and the dimer compounds. The dimer compounds in dehalogenation reaction are formed, when a compound of formula IV get reduced to form amines derivative compound which reacts with a compound of formula III.

In an embodiment, dehalogenation of halogenated benzonitrile of formula III to halogenated benzonitrile of formula II is carried out in presence of a solvent selected from water and organic solvent such as toluene, tetrahydrofuran, dioxane, acetonitrile, benzonitrile, toluene, iso-butanol, sulfolane, dimethyl formamide, xylene isomers, or like or mixture thereof.

In another embodiment, dehalogenation of halogenated benzonitrile of formula III to halogenated benzonitrile of formula II is carried out in absence of organic solvent. The absence of organic solvent in dehalogenation step eliminates the formation of by-products.

In a preferred embodiment, dehalogenation of halogenated benzonitrile of formula III to halogenated benzonitrile of formula II is carried out in water.

In a specific embodiment, 3,5-dichloro-2,4,6-trifluorobenzonitrile is dechlorinated using zinc and acetic acid to obtain 2,4,6-trifluorobenzonitrile in presence of water as solvent.

In an embodiment, dehalogenation of halogenated benzonitrile of formula III to halogenated benzonitrile of formula II is carried out using transition metal catalyst, alkanoic acid and a salt. The salt is selected from phosphate salt such as dipotassium hydrogen phosphate, ammonium dihydrogen phosphate, ammonium hydrogen phosphate, calcium dihydrogen phosphate, calcium hydrogen phosphate, calcium phosphate, potassium dihydrogen phosphate, potassium hydrogen phosphate, sodium dihydrogen phosphate, sodium hydrogen phosphate, acetates salt such as sodium acetate potassium acetate, and ammonium salt such as tetramethylammonium chloride, trioctylmethylammonium chloride, tetraphenylphosphonium chloride and bromide, tetrabutylammonium chloride, bromide or hydrates thereof.

In a specific embodiment, 3,5-dichloro-2,4,6-trifluorobenzonitrile is dechlorinated using zinc and formic acid to obtain 2,4,6-trifluorobenzonitrile in absence of organic solvent.

The impurities formed during the preparation of 2,4,6-trifluorobenzonitrile from 3,5-dichloro-2,4,6-trifluorobenzonitrile are identified as 2,6-difluorobenzonitrile, 2,4-difluorobenzonitrile and the dimer.

In another specific embodiment, 3,5-dichloro-2,4,6-trifluorobenzonitrile is dechlorinated using zinc and acetic acid in presence potassium dihydrogen phosphate to obtain 2,4,6-trifluorobenzonitrile.

In an embodiment, the purity of isolated halogenated benzonitrile of formula II is greater than 90% and preferably 95% and more preferably 98%.

In another embodiment, halogenated benzonitrile of formula II contains, impurities less than 2% and more preferably less than 1%.

The yield of halogenated benzonitrile of formula II is greater than 80% and preferably greater than 85%.

The compound of formula III may be selected from 3,5-dichloro-2,4,6-trifluorobenzonitrile, 3,5-dibromo-2,4,6-trifluorobenzonitrile, 3-bromo-5-chloro-2,4,6-trifluorobenzonitrile, 3,5-dichloro-2,4-difluorobenzonitrile, 3,5-dichloro-4-fluorobenzonitrile, 3,5-dichloro-2,6-difluorobenzonitrile or like.

The compound of formula II may be selected from 2,4,6-trifluorobenzonitrile, 2,4,6-trichlorobenzonitrile, 2,4-difluorobenzonitrile, 4-fluorobenzonitrile, 2,6-difluorobenzonitrile or like.

In an embodiment, halogenated benzonitrile of formula II is hydrogenated to obtain halogenated benzylamines of formula I.

As used herein, “hydrogenated” refers to reaction of halogenated benzonitrile with hydrogen. The hydrogenation is carried out under a hydrogen pressure in the range of 10 to 15 Kg/cm².

The hydrogenation reaction is carried out at 50° C. to 90° C. and more preferably in the range 60° C. to 80° C.

In an embodiment, the hydrogenation is carried out using a hydrogenation catalyst and solvent. The hydrogenation catalyst is selected from palladium on carbon, platinum on carbon, Raney nickel or the like. The molar ratio of catalyst may be selected in the range from 0.05 to 0.3. The Raney nickel contains nickel in the range from 70-95% and more preferably 80-90%.

In another embodiment, weight % of catalyst with respect to the compound of formula II is in the range of 1-10% and more preferably in the range of 5-10%.

The solvent for hydrogenation is alcohol selected from the group consisting of water and alcohols such as methanol, ethanol, 2-propanol, propanol, butanol, t-butanol, hexanol, pentanol or like and mixture thereof. It has been found that alcohols comprising C-3 or more are preferred as lower alcohol tend to substitute the ring halogen result into alkoxy-impurities, which proved to be very difficult to separate from the final amine by distillation.

In preferred embodiment, hydrogenation is carried out alcohol solvent selected from 2-propanol, propanol, t-butanol, hexanol and pentanol.

In a specific embodiment, hydrogenation is carried out using a catalyst Raney-nickel in the range of around 0.14 to 0.2 molar equivalents.

The halogenated benzonitrile of formula II may be used as such, molten form or as alcoholic solution and more preferably as alcoholic solution.

The ammonia is used in pure form or solution form selected from anhydrous ammonia, aqueous ammonia and alcoholic ammonia solution. The molar ratio of ammonia is selected in the range of 1-5 by mass of ammonia.

In an embodiment, hydrogenation of halogenated benzonitrile of formula II is carried out using anhydrous ammonia to obtain halogenated benzylamine of formula I. It prevents formation of azeotropic mixture of water and alcohol which need azeotropic distillation.

In an embodiment, hydrogenation catalyst and the solvent is charged and purged with anhydrous ammonia followed by continuous addition of halogenated benzonitrile of formula

II to obtain halogenated benzylamine of formula I.

In an embodiment, hydrogenation reaction is carried out in using aqueous ammonia in the range of 25% to 35%.

In a preferred embodiment, hydrogenation is carried out using ammonia in the range of 2.0 to 4.0 molar equivalents.

In an embodiment, the halogenated benzonitrile of formula II is added continuously in the reaction mixture. The addition rate of halogenated benzonitrile of formula II is selected from 0.5-2 ml/min, through HPLC pump. The addition is carried out in 10-16 hours.

In a preferred embodiment, an alcoholic solution of halogenated benzonitrile of formula II is added to a mixture of an alcohol, hydrogenation catalyst and ammonia.

It was observed by inventors of present application that mode and sequence of addition of reactant and reagent in the reaction affects the product selectivity. The continuous addition of an alcoholic solution of halogenated benzonitrile of formula II have been found to control the reaction dynamics and surprisingly improved the selectivity of halogenated benzylamine of formula I to 91-95%. The continuous addition of an alcoholic solution of halogenated benzonitrile of formula II reduces impurity formation, resulting from the dehalogenation of X₁, selected from chloro or fluoro.

The results obtained for continuous addition of an alcoholic solution of 2,4,6-trifluorobenzonitrile in the reaction mixture containing alcohol, catalyst and ammonia are given in Table 1.

The impurities formed in the hydrogenation of halogenated benzonitrile of formula II to halogenated benzylamine of formula I results from dehalogenation of X₁, selected from chloro or fluoro and dimer compounds. The dimer compounds are formed by reaction of two molecules of compound of formula I either via substitution of halogen of one molecule by second molecule of compound of formula I via NH₂-attachment or via substitution of amino group of one molecule with a compound of formula I via NH₂-attachment.

In a specific embodiment, the continuous addition of an alcoholic solution of 2,4,6-trifluorobenzonitrile controlled the reaction dynamics and improved the selectivity of 2,4,6-trifluorobenzylamine to 91-95%.

In a specific embodiment, hydrogenation is carried out by continuously adding 2,4,6-trifluorobenzonitrile to a mixture of aqueous ammonia and Raney nickel in presence of t-butanol.

In a specific embodiment, hydrogenation is carried out by continuously adding 2,4,6-trifluorobenzonitrile to a mixture of anhydrous ammonia and Raney nickel in presence of t-butanol.

In a specific embodiment, hydrogenation is carried out by continuously adding 2,4-difluorobenzonitrile to a mixture of anhydrous ammonia and Raney nickel in presence of 2-propanol.

In a specific embodiment, hydrogenation is carried out by continuously adding 2,4,6-trifluorobenzonitrile to a mixture of aqueous ammonia and Raney nickel in presence of 2-propanol.

In another embodiment of the present invention, the continuous addition is carried out using a continuous flow reactor.

The hydrogenation of 2,4,6-trifluorobenzonitrile when carried out as per the process of the present invention reduces the impurities resulting from dehalogenation of X₁ identified as 2,6-difluorobenzonitrile, 2,4-difluorobenzonitrile and dimers.

The halogenated benzylamine of formula I is isolated by filtration and distillation.

In an embodiment, present invention provides a process for preparation of 2,4,6-trifluorobenzylamine comprising the steps of:

a) dechlorination of 3,5-dichloro-2,4,6-trifluorobenzonitrile with zinc in presence of acetic acid to obtain 2,4,6-trifluorobenzonitrile; and

b) hydrogenating 2,4,6-trifluorobenzonitrile using Raney nickel and anhydrous ammonia in presence of 2-propanol to obtain 2,4,6-trifluorobenzylamine.

The purity of halogenated benzylamine of formula I is greater than 90% and preferably 95% and more preferably greater than 98%.

The halogenated benzylamine of formula I is having less than 2% of impurities and preferably less than 1%.

The alcohol solvent, hydrogenation catalyst and ammonia are recycled from hydrogenation step of present invention.

Halogenated benzylamines are isolated by any method known in the art, for example, chemical separation, extraction, acid-base neutralization, distillation, evaporation, column chromatography and filtration or a mixture thereof.

TABLE 1
Comparison study of experiments.

| | | Experiment Selectivity % | | | |
|---|---|---|---|---|---|
| | | | | Continuous addition of | |
| | | Aqueous | | 2,4,6-benzonitrile | |
| S. | | 25% | Ammonium | Aqueous | Anhydrous |
| No. | Component | ammonia | acetate | ammonia | ammonia |
| 1 | 2,4,6- | 45-50 | 75-85 | 91-95 | 91-94 |
| | trifluoroben- | | | | |
| | zylamine | | | | |
| 2 | Alkoxy | 30-35 | 0 | 0-1 | 0-1 |
| | impurity | | | | |
| 3 | Dimer | 14-15 | 13-15 | 0-2 | 0-2 |
| | impurity | | | | |

The product is isolated by any method known in the art, for example, chemical separation, extraction, acid-base neutralization, distillation, evaporation, column chromatography and filtration or a mixture thereof.

The completion of the reaction may be monitored by any one of chromatographic techniques such as thin layer chromatography (TLC), high pressure liquid chromatography (HPLC), ultra-pressure liquid chromatography (UPLC), Gas chromatography (GC), liquid chromatography (LC) and alike.

The reagents and raw material used for present invention may be prepared or obtained commercially.

Unless stated to the contrary, any of the words “comprising”, “comprises” and includes mean “including without limitation” and shall not be construed to limit any general statement that it follows to the specific or similar items or matters immediately following it.

Embodiments of the invention are not mutually exclusive, but may be implemented in various combinations. The described embodiments of the invention and the disclosed examples are given for the purpose of illustration rather than limitation of the invention as set forth in the appended claims.

The following examples are given by way of illustration and therefore should not be construed to limit the scope of the present invention.

### EXAMPLES

### Example 1: Preparation of 2,4,6-trifluorobenzonitrile

Zinc powder (43.49 gm), water (350 gm) and 3,5-Dichloro-2,4,6-trifluorobenzonitrile (50 gm) were charged into 1000 ml glass reactor assembly under stiffing. Reaction mass was heated to 75° C. under stirring. Formic acid (30.5 gm) was slowly added to reaction mass at 75° C. Reaction is monitored by Gas chromatography. After completion of reaction, the mass was cooled to 35-38° C. and dichloromethane (300 gm) was added to reaction mass. Filtered the reaction mass under vacuum and layer were separated. Dichloromethane was recovered from organic layer and residue was fractionally distilled.

Yield: 85%; Purity: 99.5%.

### Example 2: Preparation of 2,4,6-trifluorobenzonitrile

Zinc powder (73 gm), water (700 gm), 3,5-Dichloro-2,4,6-trifluorobenzonitrile (100 gm) and potassium dihydrogen phosphate (2 gm) were charged into 2-litre glass round bottom flask under stiffing. Reaction mass was heated to 70-80° C. under stiffing. Acetic acid (80 gm) was slowly added to reaction mass in 2-3 hours at 70-80° C. Reaction was monitored by GC analysis. After completion of reaction, the mass was cooled to 35-38° C. and dichloromethane (600 gm) was added. Filtered the reaction mass under vacuum and layer were separated. Dichloromethane was recovered from organic layer and residue was fractionally distilled.

Yield: 70%; Purity (GC): 99.5%.

### Example 3: Preparation of 2,4,6-trifluorobenzonitrile

Zinc powder (43.38 gm), water (350 gm) and 3,5-Dichloro-2,4,6-trifluorobenzonitrile (50 gm) were charged into 1000 ml glass reactor assembly under stiffing. Reaction mass was heated to 72° C. under stiffing. Trifluoroacetic acid (75.60 gm) was added in to the reaction mass at 72° C. Reaction was monitored by Gas chromatography. After completion of reaction, the mass was cooled to 35-38° C. and dichloromethane (600 gm) was added to reaction mass. Filtered the reaction mass under vacuum and layer were separated. Dichloromethane was recovered from organic layer and residue was fractionally distilled.

Yield: 80%; Purity: 99.5%.

### Example 4: Preparation of 2,4,6-trifluorobenzonitrile

Zinc powder (6.14 gm), water (4.7 gm), and 3-chloro-2,4,6-trifluorofluorobenzonitrile (10 gm) were charged into 250 ml glass reactor assembly under stiffing. Reaction mass was heated to 70° C. under stiffing. Acetic acid (6.27 gm) was slowly added to reaction mass at 70° C. Reaction was monitored after 3 hours by Gas chromatography. After completion of reaction, the mass was cooled to 35-38° C. and dichloromethane (60 gm) was added to reaction mass. Filtered the reaction mass under vacuum and layer were separated. Dichloromethane was recovered from organic layer and residue was fractionally distilled.

Reaction composition shows:

2,4,6-trifluorobenzonitrile—60.0%

3-chloro-2,4,6-trifluorobenzonitrile-14.72%

### Example 5: Preparation of 2,4,6-trifluorobenzonitrile

Zinc powder (8.70 gm), water (70 gm), toluene (40.0 gm), tetraphenylphosphonium bromide (1.83 gm) and 3,5-Dichloro-2,4,6-trifluorobenzonitrile (10.0 gm) were charged into 250 ml glass reactor assembly under stirring. Reaction mass was heated to 70° C. under stiffing. Acetic acid (8.0 gm) was slowly added to reaction mass at 70° C. Reaction was monitored by Gas chromatography after 2 hours.

Reaction analysis shows:

2,4,6-trifluorobenzonitrile—43.93%

3-chloro-2,4,6-trifluorobenzonitrile—27.49%

3,5-dichloro-2,6-difluorobenzonitrile—16.34%

### Example 6: Preparation of 2,4,6-trifluorobenzonitrile

Zinc powder (43.51 gm), water (350.0 gm), toluene (34.0 gm), trioctylmethylammonium chloride (0.690 gm) and 3,5-Dichloro-2,4,6-trifluorobenzonitrile (50.0 gm) were charged into 1000 ml glass reactor assembly under stirring. Reaction mass was heated to 72° C. under stiffing. Acetic acid (40.0 gm) was slowly added to reaction mass at 72° C. Reaction was monitored by Gas chromatography after complete addition of acetic addition.

Reaction analysis shows:

2,4,6-trifluorobenzonitrile—21.278%

3-chloro-2,4,6-trifluorobenzonitrile—39.138%

3,5-dichloro-2,6-difluorobenzonitrile—16.222%

### Example 7: Preparation of 2,4,6-trifluorobenzonitrile

Zinc powder (43.50 gm), water (350.0 gm), toluene (34.0 gm), tetrabutylammonium bromide (0.544 gm) and 3,5-Dichloro-2,4,6-trifluorobenzonitrile (50.0 gm) were charged into 250 ml glass reactor assembly under stirring. Reaction mass was heated to 72° C. under stiffing. Acetic acid (40.0 gm) was slowly added to reaction mass at 72° C. Reaction was monitored by Gas chromatography after complete addition of acetic addition.

Reaction analysis shows:

2,4,6-trifluorobenzonitrile—18.934%

3-chloro-2,4,6-trifluorobenzonitrile-60.192%

3,5-dichloro-2,6-difluorobenzonitrile—12.576%

### Example 8: Preparation of 2,4,6-trifluorobenzonitrile

Zinc powder (21.57 gm), water (174.20 gm), toluene (16.59 gm), tetramethylammonium chloride (0.50 gm) and 3,5-Dichloro-2,4,6-trifluorobenzonitrile (25.0 gm) were charged into 250 ml glass reactor assembly under stiffing. Reaction mass was heated to 72° C. under stiffing. Acetic acid (19.80 gm) was slowly added to reaction mass at 72° C. Reaction was monitored by Gas chromatography after complete addition of acetic addition.

Reaction analysis shows:

2,4,6-trifluorobenzonitrile—60.030%

3-chloro-2,4,6-trifluorobenzonitrile—27.913%

3,5-dichloro-2,6-difluorobenzonitrile—4.720%

### Example 9: Preparation of 2,4,6-trifluorobenzonitrile. (Comparative Example)

Zinc powder (12.0 gm), water (8 gm) and 3,5-Dichloro-2,4,6-trifluorobenzonitrile (20 gm) were charged into 250 ml glass RBF assembly under stiffing. Reaction mass was heated to 70° C. under stiffing. Sulfuric acid (13.17 gm) was slowly added to reaction mass at 70° C. Reaction was monitored after 4 hours by Gas chromatography. After completion of reaction, the mass was cooled to 35-38° C. and dichloromethane (60 gm) is added to reaction mass. Filtered the reaction mass under vacuum and layer were separated. Dichloromethane was recovered from organic layer and residue was fractionally distilled.

Reaction analysis shows

2,4,6-trifluorobenzonitrile—13.43%

3-chloro-2,4,6-trifluorobenzonitrile-20.76%

3,5-Dichloro-2,4,6-trifluorobenzonitrile-4.55%

### Example 10: Preparation of 2,4,6-trifluorobenzonitrile. (Comparative Example

Zinc powder (11.62 gm), water (21.62 gm), benzonitrile (100 ml) and 3,5-Dichloro-2,4,6-Trifluorobenzonitrile (10 gm) were charged into 250 ml glass reactor assembly under stiffing. Reaction mass was heated to 80° C. under stiffing. Phosphoric acid (17.42 gm) was slowly added to reaction mass at 80° C. Reaction was monitored after 1 hours by Gas chromatography. After completion of reaction, the mass is cooled to 35-38° C. and dichloromethane (60 gm) was added to reaction mass. Filtered the reaction mass under vacuum and layer were separated. Dichloromethane was recovered from organic layer and residue was fractionally distilled.

Reaction composition shows

2,4,6-trifluorobenzonitrile—1.423%

3-chloro-2,4,6-trifluorobenzonitrile-27.097%

3,5-Dichloro-2,4,6-trifluorobenzonitrile-53.07%

### Example 11: Preparation of 2,4,6-trifluorobenzyl Amine

Raney Nickel (8 gm), 2-propanol (550 gm) and 25% aqueous ammonia (174 gm) were charged into a reactor. The reactor was flushed once with nitrogen and then with hydrogen. The reaction mixture was heated to 60-80° C. Hydrogen was charged in reactor at 60-80° C. continuously to 10 kg/cm². A solution of 2,4,6-trifluorobenzonitrile (18% in 2-propanol) was charged in the reactor using a HPLC pump at a rate of 0.5-2 ml/min in 10-12 hours. After completion of the reaction, the reaction mass was cooled to 20-25° C. and residual pressure was released. Reaction mass was filtered, washed with 2-propanol (200 gm). The filtrate was concentrated through recovery of ammonia, azeotropic removal of water with 2-propanol. The bottom mass was then purified by distillation under reduced pressure.

Yield (%): 91%; Purity (by GC): 99.1%

### Example 12: Preparation of 2,4,6-trifluorobenzyl Amine

Raney Nickel (8 gm) and 2-propanol (550 gm) were charged into a reactor. The reactor was flushed once with nitrogen and then with hydrogen. The reaction mixture was heated to 65-75° C. Hydrogen was charged in reactor at 65-75° C. continuously to 10 kg/cm². Anhydrous ammonia was charged in the reactor at 65-75° C. A solution of 2,4,6-trifluorobenzonitrile (15% in 2-propanol) was charged in the reactor using a HPLC pump at a rate of 0.5-2 ml/min in 10-12 hours. After completion of the reaction, the reaction mass was cooled to 20-25° C. and residual pressure was released. Reaction mass was filtered, washed with 2-propanol (200 gm). The filtrate was concentrated and purified by distillation under reduced pressure.

Yield (%): 94%; Purity (by GC): 97%

### Example 13: Preparation of 2-fluorobenzyl Amine

Raney Nickel (10.3 gm), 2-propanol (420 gm) and 25% aqueous ammonia (226 gm) were charged into a reactor. The reactor was flushed once with nitrogen and then with hydrogen. The reaction mixture was heated to 60-80° C. Hydrogen was charged in reactor at 60-80° C. continuously to 10 kg/cm². A solution of 2-fluorobenzonitrile (18%-20% in 2-propanol) was charged in the reactor using a HPLC pump at a rate of 0.5-2 ml/min in 15-16 hours. After completion of the reaction, the reaction mass was cooled to 20-25° C. and residual pressure was released. Reaction mass was filtered, washed with 2-propanol twice (100 gm). The filtrate was concentrated through recovery of ammonia, azeotropic removal of water with 2-propanol. The bottom mass was then purified by distillation under reduced pressure.

Yield (%): 92%; Purity (by GC): 99.3%

### Example 14: Preparation of 4-fluorobenzyl Amine

Raney Nickel (10.3 gm), 2-propanol (420 gm) and 25% aqueous ammonia (226 gm) were charged into a reactor. The reactor was flushed once with nitrogen and then with hydrogen. The reaction mixture was heated to 60-80° C. Hydrogen was charged in reactor at 60-80° C. continuously to 10 kg/cm². A solution of 4-fluorobenzonitrile (18%-20% in 2-propanol) was charged in the reactor using a HPLC pump at a rate of 0.5-2 ml/min in 14-16 hours. After completion of the reaction, the reaction mass was cooled to 20-25° C. and residual pressure was released. Reaction mass was filtered, washed with 2-propanol twice (100 gm). The filtrate was concentrated through recovery of ammonia, azeotropic removal of water with 2-propanol. The bottom mass was then purified by distillation under reduced pressure.

Yield (%): 93%; Purity (by GC): 99.2%

### Example 15: Preparation of 2,6-difluorobenzyl Amine

Raney Nickel (9.1 gm), 2-propanol (490 gm) and 25% aqueous ammonia (195 gm) were charged into a reactor. The reactor was flushed once with nitrogen and then with hydrogen. The reaction mixture was heated to 60-80° C. Hydrogen was charged in reactor at 60-80° C. continuously to 10 kg/cm². A solution of 2,6-difluorobenzonitrile (18% in 2-propanol) was charged in the reactor using a HPLC pump at a rate of 0.5-2 ml/min in 12-13 hours. After completion of the reaction, the reaction mass was cooled to 20-25° C. and residual pressure was released. Reaction mass was filtered, washed with 2-propanol (200 gm). The filtrate was concentrated through recovery of ammonia, azeotropic removal of water with 2-propanol. The bottom mass was then purified by distillation under reduced pressure.

Yield (%): 90%; Purity (by GC): 99%

### Example 16: Preparation of 2,4-difluorobenzyl Amine

Raney Nickel (9.1 gm), 2-propanol (490 gm) and 25% aqueous ammonia (195 gm) were charged into a reactor. The reactor was flushed once with nitrogen and then with hydrogen. The reaction mixture was heated to 60-80° C. Hydrogen was charged in reactor at 60-80° C. continuously to 10 kg/cm². A solution of 2,4-difluorobenzonitrile (18% in 2-propanol) was charged in the reactor using a HPLC pump at a rate of 0.5-2 ml/min in 12-14 hours. After completion of the reaction, the reaction mass was cooled to 20-25° C. and residual pressure was released. Reaction mass was filtered, washed with 2-propanol (200 gm). The filtrate was concentrated through recovery of ammonia, azeotropic removal of water with 2-propanol. The bottom mass was then purified by distillation under reduced pressure.

Yield (%): 90%; Purity (by GC): 99.3%

### Example 17: Preparation of 2,4,6-trichlorobenzyl Amine

Raney Nickel (6.1 gm), 2-propanol (660 gm) and 25% aqueous ammonia (135 gm) were charged into a reactor. The reactor was flushed once with nitrogen and then with hydrogen. The reaction mixture was heated to 60-80° C. Hydrogen was charged in reactor at 60-80° C. continuously to 10 kg/cm². A solution of 2,4,6-trichlorobenzonitrile (18% in 2-propanol) was charged in the reactor using a HPLC pump at a rate of 0.5-2 ml/min in 8-10 hours. After completion of the reaction, the reaction mass was cooled to 20-25° C. and residual pressure was released. Reaction mass was filtered, washed with 2-propanol (200 gm). Ammonia, 2-propanol and water was recovered from filtrate. The bottom mass was then purified by distillation under reduced pressure.

Yield (%): 75%; Purity (by GC): 99.5%

### Example 18: Preparation of 2-chlorobenzyl Amine

Raney Nickel (9.13 gm), 2-propanol (485 gm) and 25% aqueous ammonia (200 gm) were charged into a reactor. The reactor was flushed once with nitrogen and then with hydrogen. The reaction mixture was heated to 60-80° C. Hydrogen was charged in reactor at 60-80° C. continuously to 10 kg/cm². A solution of 2-chlorobenzonitrile (18% in 2-propanol) was charged in the reactor using a HPLC pump at a rate of 0.5-2 ml/min in 11-13 hours. After completion of the reaction, the reaction mass was cooled to 20-25° C. and residual pressure was released. Reaction mass was filtered, washed with 2-propanol (200 gm). Ammonia, 2-propanol and water was recovered from filtrate. The product was purified by distillation at 50-60° C. under reduced pressure.

Yield (%): 90%; Purity (by GC): 99%

### Example 19: Preparation of 4-chlorobenzyl Amine

Raney Nickel (9.13 gm), 2-propanol (485 gm) and 25% aqueous ammonia (200 gm) were charged into a reactor. The reactor was flushed once with nitrogen and then with hydrogen. The reaction mixture was heated to 60-80° C. Hydrogen was charged in reactor at 60-80° C. continuously to 10 kg/cm². A solution of 4-chlorobenzonitrile (18% in 2-propanol) was charged in the reactor using a HPLC pump at a rate of 0.5-2 ml/min in 10-12 hours. After completion of the reaction, the reaction mass was cooled to 20-25° C. and residual pressure was released. Reaction mass was filtered, washed with 2-propanol (200 gm). Ammonia, 2-propanol and water was recovered from filtrate. The product was purified by distillation under reduce pressure.

Yield (%): 80%; Purity (by GC): 99.2%

### Example 20: Preparation of 2,6-dichlorobenzyl Amine

Raney Nickel (7.3 gm), 2-propanol (590 gm) and 25% aqueous ammonia (160 gm) were charged into a reactor. The reactor was flushed once with nitrogen and then with hydrogen. The reaction mixture was heated to 60-80° C. Hydrogen was charged in reactor at 60-80° C. continuously to 10 kg/cm². A solution of 2,6-dichlorobenzonitrile (18% in 2-propanol) was charged in the reactor using a HPLC pump at a rate of 0.5-2 ml/min in 10-12 hours. After completion of the reaction, the reaction mass was cooled to 20-25° C. and residual pressure was released. Reaction mass was filtered, washed with 2-propanol (200 gm). Ammonia, 2-propanol and water was recovered from filtrate. The product was purified by distillation under reduce pressure.

Yield (%): 75%; Purity (by GC): 99.6%

### Example 21: Preparation of 2,4-dichlorobenzyl Amine

Raney Nickel (7.3 gm), 2-propanol (590 gm) and 25% aqueous ammonia (174 gm) were charged into a reactor. The reactor was flushed once with nitrogen and then with hydrogen. The reaction mixture was heated to 60-80° C. Hydrogen was charged in reactor at 60-80° C. continuously to 10 kg/cm². A solution of 2,4-dichlorobenzonitrile (18% in 2-propanol) was charged in the reactor using a HPLC pump at a rate of 0.5-2 ml/min in 10-12 hours. After completion of the reaction, the reaction mass was cooled to 20-25° C. and residual pressure was released. Reaction mass was filtered, washed with 2-propanol (200 gm). Ammonia, 2-propanol and water was recovered from filtrate. The product was purified by distillation under reduce pressure.

Yield (%): 78%; Purity (by GC): 99.4%

### Example 22: Preparation of 2,4-difluorobenzyl Amine

Raney Nickel (7.3 gm), 2-propanol (590 gm) were charged into a reactor. The reactor was flushed once with nitrogen and then with hydrogen. The reaction mixture was heated to 70° C. Hydrogen was charged in reactor at 70-80° C. continuously to 10 kg/cm². Anhydrous ammonia was charged in the reactor at 70-80° C. A solution of 2,4-difluorobenzonitrile (20% in 2-propanol) was charged in the reactor using a HPLC pump at a rate of 0.5-2 ml/min in 10-12 hours. After completion of the reaction, the reaction mass was cooled to 20-25° C. and residual pressure was released. Reaction mass was filtered, washed with 2-propanol (200 gm). The filtrate was concentrated and purified by distillation under reduce pressure.

Yield (%): 75-78%; Purity (by GC): >99%

1. A process for preparation of halogenated benzylamine of formula I, 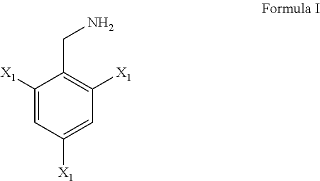
wherein, X₁ is selected from hydrogen, chloro and fluoro, provided at least one X₁ is chloro or fluoro,
said process comprising the steps of:
a) selective dehalogenation of halogenated benzonitrile of formula III, 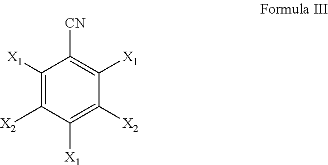
wherein, X₁ is selected from hydrogen, chloro and fluoro, provided at least one X₁ is chloro or fluoro; X₂ is selected from chloro or bromo;
using a transition metal catalyst in presence of an alkanoic acid to obtain a halogenated benzonitrile of formula II, 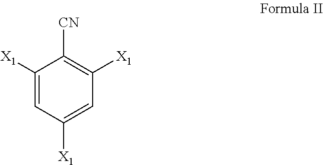
wherein X₁ is selected from hydrogen, chloro or fluoro, provided at least one X₁ is chloro or fluoro; and
b) hydrogenating halogenated benzonitrile of formula II using hydrogenating catalyst and ammonia in presence of a solvent to obtain a halogenated benzylamine of formula I.

2. A process for preparation of halogenated benzylamine of formula I comprising a step of hydrogenating halogenated benzonitrile of formula II being carried out by continuous addition of halogenated benzonitrile of formula II to a mixture of hydrogenating catalyst, ammonia and solvent.

3. The process as claimed in claim 1, wherein step a) is carried out using a transition metal catalyst selected from copper, zinc, and zinc/copper alloy.

4. The process as claimed in claim 1, wherein said alkanoic acid is selected from formic acid, acetic acid, and trifluoroacetic acid.

5. The process as claimed in claim 1, wherein step a) is carried out in absence of an organic solvent at a temperature of 70° C. to 90° C.

6. The process as claimed in claim 1, wherein the step b) is carried out by continuous addition of halogenated benzonitrile of formula II to a mixture of hydrogenating catalyst and ammonia in presence of solvent.

8. The process as claimed in claim 1, wherein the hydrogenation is carried out using a hydrogenating catalyst selected from palladium on carbon, platinum on carbon, and Raney nickel.

9. The process as claimed in claim 1, wherein the hydrogenation is carried out in presence of a solvent selected from 2-propanol, propanol, butanol, t-butanol, hexanol, and pentanol or mixture thereof.

10. The process as claimed in claim 1, wherein ammonia is selected from anhydrous ammonia, aqueous ammonia and alcoholic ammonia solution.
